SEQUENCE LISTING (1 sequence)

SEQ ID NO: 1 - DNA, 15 bp, Artificial Sequence
  nucleic acid sequence for the locking for a nucleic acid
cttcaactggcagct

(19) United States
(12) Patent Application Publication
(10) Pub. No.: US 2019/0000886 A1
(43) Pub. Date: Jan. 3, 2019

(54) METHOD OF TREATMENT OF OSTEOCHONDRAL DEFECTS

(71) Applicant: Duncan Ross, Miami Beach, FL (US)

(72) Inventor: Duncan Ross, Miami Beach, FL (US)

(21) Appl. No.: 16/003,368

(22) Filed: Jun. 8, 2018

Related U.S. Application Data
(63) Continuation-in-part of application No. PCT/US2016/022629, filed on Mar. 16, 2016.
(60) Provisional application No. 62/517,847, filed on Jun. 9, 2017.

(51) Int. Cl. A61K 35/28; A61K 35/50; A61K 35/36; A61K 35/33; A61K 35/35; A61K 35/30; A61K 35/22; A61K 35/42; A61K 35/32; A61K 35/407; A61K 38/18; A61K 31/573; A61K 31/661; A61K 31/19; A61P 19/02

(52) U.S. Cl. CPC A61K 35/28; A61P 19/02; A61K 35/36; A61K 35/33; A61K 35/35; A61K 35/30; A61K 35/22; A61K 35/42; A61K 35/32; A61K 35/407; A61K 38/1825; A61K 38/1841; A61K 38/18; A61K 31/573; A61K 31/661; A61K 31/19; A61K 35/50

(57) ABSTRACT

Method and pharmaceutical compositions for enhancing osteochondral tissue, so that osteochondral defects may be repaired by the body and/or prevented or lessened. The methods primarily include administering into a joint cavity, in doses not more frequently than daily for up to 13 days, an effective amount of anti-inflammatory cell growth pharmaceutical composition including cell secretory microvesicles derived from placental cotyledon derived mesenchymal stem cells that secrete growth factors; and administering into the joint cavity on day 14 (and as appropriate thereafter) an effective amount of pharmaceutical composition for cell differentiation and growth including chondrocyte derived exosomes.

### CROSS REFERENCE TO RELATED APPLICATIONS

This application is a continuation of pending Provisional Application No. 62/517,847 filed 9 Jun. 2017, and claims the benefit of the filing date thereof. This application also a C-I-P and claims priority benefit of an International Application Number PCT/US2016/022629 filed on 16 Mar. 2016.

### FEDERALLY SPONSORED RESEARCH AND DEVELOPMENT

Not applicable.

### MICROFICHE APPENDIX

Not applicable.

### BACKGROUND OF THE INVENTION

### Field

The present disclosure relates to methods of treatment for osteochondral defects involving administration of membrane-enclosed vesicles according to a defined time course and using a color scheme for proper utilization to avoid physician error.

### Background

Mesenchymal stem cells are multipotent cells present throughout the tissues of the body that possess a secretory component consisting of membrane enclosed vesicles that contain anti-inflammatory compounds as well as growth factors in the form of proteins and nucleic acids.

Mesenchymal stem cells (MSC) are defined by their ability to differentiate into cells of a bone, cartilage, or adipose phenotype in the developing animal. Adult cells of either an autologous or allogeneic origin have been employed to treat osteochondral defects in adults with limited success. Treatments in the clinic currently consist of an initial autologous cell implantation with a non-cellular ‘booster’ application usually weeks to months after the initial treatment. Booster compositions generally consist of platelet rich plasma which is known to cause inflammation. While inflammation does cause recruitment of cells to an area, it also causes a phenotypic change in mesenchymal stem cells and results in the improper deposition of cartilage in the case of osteochondral defects (non-hyaline cartilage) or epithelial wounds (scar) etc. The persistence and health of implanted cells is also not addressed in-situ by the simple use of reagents that are used to elicit an inflammatory response.

Cell secreted membrane-enclosed vesicles perform a variety of functions in a living organism and can in many cases elicit the same functional result as a cell. For example, membrane-enclosed vesicles are involved in transportation of cellular materials, enzyme storage, metabolism and cell programming and can be derived from mesenchymal stem cells, chondrocytes, or other cells. Membrane vesicles are found in amniotic fluid, serum, milk, saliva, etc. For example, if a subject experiences inflammation of organs, e.g., lungs and skin, the membrane-enclosed vesicles from the MSC of a healthy donor may through normal cellular function provide some range of anti-inflammatory and regenerative effect.

The present disclosure is directed towards a novel method and schedule of administering membrane-enclosed vesicles with various provenances to a subject in order to provide a therapeutic benefit for osteochondral defects.

Amniotic fluid [AF] serves a number of roles towards the developing fetus and contains a defined composition of growth factors, the quantities of which will vary from donor to donor. AF consists of the mother's epithelial cells, a diminishing number of mesenchymal stem cells as the gestation progresses, hyaluronic acid to cushion the fetus and growth factors both free and contained within membrane enclosed vesicles. Amniotic fluid has been used for centuries to treat maladies and is currently erroneously referred to as a ‘stem cell treatment’ in cases of joint based pain. Patient relief in AF treated cases is due more to the anti-inflammatory component of the cells than a regenerative or de-novo cartilage creation function as demonstrated in clinical trials wherein no cartilage regeneration has been noted but patient pain scores have fallen.

It is an object of the invention to provide timing and methods of use and production that will address the above shortcomings of cell persistence and phenotype. The invention concerns a novel combination of steps employing the timed harvest of autologous cells for treatment of osteo-arthritis (OA), cell factor administration from multiple origins to provide a full growth stimulating factor component in a non-inflammatory setting, and visual differentiation of the solutions involved.

### SUMMARY OF THE INVENTION

The present invention provides a timing of administration of cell factor compositions obtained from naturally occurring cell microvesicle secretions of juvenile origin. The use of these cell secretions herein provide a supportive, growth, and cell differentiation capacity to older adult cells that have lost the capacity to direct their own self differentiation.

Autologous cell therapies for osteochondral defects do not address the lack of growth factors and nutrients in the synovial fluid into which the cells are implanted. Nutrition is provided to outer layers of cartilage via diffusion. Persistence of a non-vascularized cell in a fluid which lacks the necessary components is on the order of days, which precludes the development of properly deposited hyaline cartilage. Further, the growth rate of a cell in an incubated space such as the knee is such that the cell must receive a specific sequence of growth factors in order to persist and expand.

Osteochondral pain and defects stem from multiple causes. Pain is associated with on-going inflammation due to tissue damage as a cause of loss of cartilage. Cartilage repair by MSC infusion in an inflammatory environment results in the deposition of fibrocartilage scar tissue, rather than the naturally occurring and preferable hyaline cartilage. Therefore, a two tiered approach is described herein. First, and in contrast to prior art, inflammation must be controlled to maintain the multipotent phenotype of donor cells when infused. Second, and on a timing outlined in this disclosure, a phenotypic differentiation of mesenchymal stem cells to cells of a chondrocyte phenotype must take place after 14 days of growth, and not before.

Cell secretions in the form of microvesicles carry growth factors and a protein milieu that is distinct from cell to cell. The present invention uses cell secretory microvesicles derived from placental cotyledon derived mesenchymal stem cells that secrete a growth factor component, in concert with a phase 2 (Day 14) growth factor component from chondrocyte derived exosomes, to treat osteochondral defects and osteoarthritis. To further the anti-inflammatory action of the first step of treatment, placental derived microvesicles are combined with cell free amniotic fluid, which provides a complimentary but dissimilar growth factor profile which can alternately be used to enhance the preliminary anti-inflammatory action in the synovial joint environment. Amniotic fluid has been used for centuries for its anti-microbial and skin replacement capabilities, and its addition to surgical protocols is warranted in almost every case.

It is known to those versed in the arts that MSC can persist for 7 days in cell culture within an initial fluid formulated to provide nutrients and growth factors. After this period the cells will exhaust the available resources or otherwise contaminate the system with cellular waste. In order to provide sufficient time for cell growth to occur and inflammation to be arrested, implanted cells or resident cells that have been induced to grow should be given a period of two weeks to expand in situ. During these two weeks, the cells will require placental exosome derived growth factors, including VEGF and PDGF for neovascularization and TGFB3 for anti-inflammatory function. They will also benefit from TNFRI and II for anti-inflammatory function and HGF and PDGF from amniotic fluid to induce growth (FIG. 2). After 14 days of growth and anti-inflammatory preparation, the cells are then ready for a FGF1 through FGF9 heavy growth factor milieu as obtained from chondrocytes in culture.

In concert with the above treatment, the growth factors should be targeted to the area of damage by using a fibrin gel as described in US patent application 20150182558. This method has been demonstrated to improve cartilage defects in preclinical literature (Zhu 2017 Nanoscale) and fibrin has been shown to induce MSC to a chondrocyte phenotype.

### BRIEF DESCRIPTION OF THE DRAWINGS

FIG. 1 consists of a schematic of timings of implantation.

FIG. 2 consists of a growth and inflammatory factor readout of the proposed microvesicles from amniotic fluid and placental cotyledon derived mesenchymal stem cells.

FIG. 3 shows a nucleic acid sequence for the locking for a nucleic acid (SEQUENCE ID NO: 1).

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification, the singular forms “a,” “an,” and “the” include plural referents unless the content clearly dictates otherwise. It should also be noted that the term “or” is generally employed in its sense including “and/or” unless the content clearly dictates otherwise.

As used herein, the term “membrane-enclosed vesicle” refers to extracellular or intracellular organelle enclosed by a lipid bilayer membrane. The membrane-enclosed vesicle may be isolated from a human or non-human cell, or may be simply synthesized or manufactured. The membrane-enclosed vesicle encapsulates various bio-molecules, such as proteins, growth factors, RNA, DNA, and the like. Non-limiting examples of membrane-enclosed vesicle includes, exosome, endosome, microvesicle, liposome, lysosome, and the like.

As used herein, the term “microvesicle” refers to a type of membrane-enclosed vesicle, derived from fragments of plasma membrane.

As used herein, the term “endosome” refers to a type of intracellular membrane-enclosed vesicle involved in cellular digestion. Endosome as used herein is not limited to any one particular type of intracellular vesicle or to any one particular stage of intracellular digestion. Endosome as used herein is meant to include, but are not limited to, early endosomes, late endosomes, and recycling endosomes.

As used herein, the term “exosome” refers to a type of extracellular membrane-enclosed vesicle, which contains molecular constituents of the cell in which it was secreted from.

As used herein, the term “growth factors” refers to a peptide or protein that stimulates the growth, differentiation, proliferation, and/or healing of cells via interaction with one or more specific cell surface receptor.

As used herein, a “subject” may be a human, non-human primate, mammal, rat, mouse, cow, horse, pig, sheep, goat, dog, cat and the like. The subject may be suspected of having or at risk for having diseases, such as inflammatory diseases and/or conditions, neurodevelopmental disorders, alcohol-induced disorders, and/or osteoporosis or other osteochondral defects.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event or circumstances may or may not occur, and that the description includes instances where said event or circumstance occurs and instances in which it does not. For example, “pharmaceutical composition comprising optional excipient” means that the excipient may or may not be present in said pharmaceutical composition.

“Exogenous” means relating to originating from outside of the original microvesicle, cell or organism of use. Exogenous compounds can be physically added to the microvesicle, cell or organism of use. “Exogenous biomolecules” relates to biomolecules that originate outside of the organism, cell and/or membrane-enclosed vesicle of use. “Exogenous biomolecules” are thus biomolecules that can be physically added to the organism, cell or membrane-enclosed vesicle, or introduced by recombinant DNA techniques. For example, exogenous DNA is DNA that introduces new characters to the organism, cell and/or membrane-enclosed vesicle that was not present previously, or creates proteins that were not present previously to the organism, cell and/or membrane-enclosed vesicle.

“Pharmaceutically acceptable carrier” or “pharmaceutically acceptable diluent” or “pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

A “pharmaceutical composition” refers to a formulation of a natural and/or synthesized compound of the invention and a medium generally accepted in the art for the delivery of the biologically active compound to mammals, e.g., humans. Such a medium includes all pharmaceutically acceptable carriers, diluents or excipients therefore.

An “effective amount” refers to a therapeutically effective amount or a prophylactically effective amount. A “therapeutically effective amount” refers to an amount effective, at dosages and for periods of time necessary, to achieve the desired therapeutic result, such as repair of damaged cartilage or other joint tissue, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as maintenance of tissue, increased life span, increased life expectancy or prevention of the progression of osteochondral defects. Typically, a prophylactic dose is used in subjects prior to or at an earlier stage of disease, so that a prophylactically effective amount may be less than a therapeutically effective amount.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Treatment Method

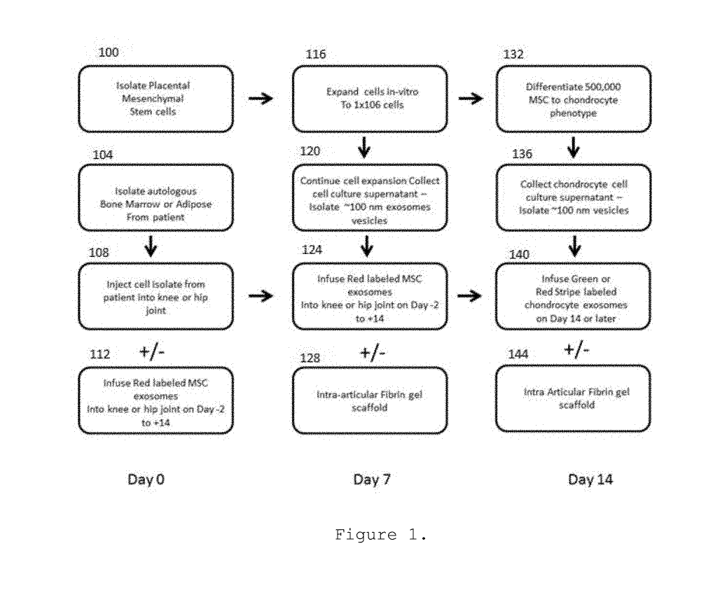

FIG. 1 shows a schematic of timings of treatment implantation. In one embodiment of this invention, the treatment method comprises the administration of a therapeutically effective amount of autologous cells comprising between 30,000 and 500,000 132 autologous CD90⁺CD105⁺CD73⁺MSC obtained from the patient via one of many harvest routes, either adipose 104, bone marrow 104, or other source of cells. Adipose samples of 60-120 cc 104 are harvested from the patient to obtain both mesenchymal stem cells as well as extra cellular matrix.

In one embodiment, Solution A (the initial anti-inflammatory cell growth solution) can be modified with a locked nucleic acid to enhance its anti-inflammatory effect, for example, including the sequence of +C+T+T+C+A+A+C+T+G+G+C+A+G+C+T.

FIG. 1 treatment timing is explained in the following description. On Day 0, first isolate Placental Mesenchymal Stem Cells (MSC) 100. Also on Day 0, isolate autologous bone marrow or Adipose from the patient 104. Then inject the isolated bone marrow or Adipose into the patient's knee or hip joint 108. As needed, infuse Red labelled MSC exosomes into the patient's knee or hip joint Day−2 to +14. 112

On day 7, expand the cells in-vitro to 1×106 cells from the isolated placental MSC 116. Then Continue cell expansion to collect cell culture supernatant and isolate ˜100 nm exosomes vesicles 120. Then infuse Red labelled MSC exosomes into patient's knee or hip joint on Day−7 to +14 (after the injection of isolated autologous bone marrow or adipose into patient's knee or hip joint) 124. As needed, stromal vascular fraction is encapsulated in a fibrin gel prior to implantation into the intra-articular space 128.

On Day 14, differentiate 500,000 MSC to chondrocyte phenotype 132. Then collect chondrocyte cell culture supernatant and isolate ˜100 nm vesicles 136. Then infuse Green or Red Stripe labelled chondrocyte exosomes on Day 14 or late (after infusion of Red labelled MSC exosomes into the knee or hip joint) 140. As needed, stromal vascular fraction is encapsulated in a fibrin gel prior to implantation into the intra-articular space 144.

In one embodiment of the present disclosure, the whole fat will be pressed to remove excess oil prior to infusion into the articular space and constitute the donor cells.

In one embodiment of the present disclosure, stromal vascular fraction via collagenase digestion of whole adipose or filtration of whole fat will be obtained in the form of a single cell suspension and infused into the intra-articular space.

In one embodiment of the present disclosure, on day 7, stromal vascular fraction will be encapsulated in a fibrin gel prior to implantation into the intra-articular space 128.

In one embodiment of the present disclosure, the autologous cellular product obtained on Day 0 of the treatment will be co-incubated with a stimulatory solution that will aid in engraftment and expansion of the newly transplanted cells in situ. In one embodiment of the invention, the day 0 stimulatory Solution A is comprised of microvesicles obtained from the supernatant of cultured placental cotyledon derived mesenchymal stem cells 100 or other inflammation suppressing cells. These microvesicles can be obtained from any type of cell, including, fetal, neonatal, juvenile or adult allogeneic or autologous cells. Alternatively, this solution can consist of amniotic fluid.

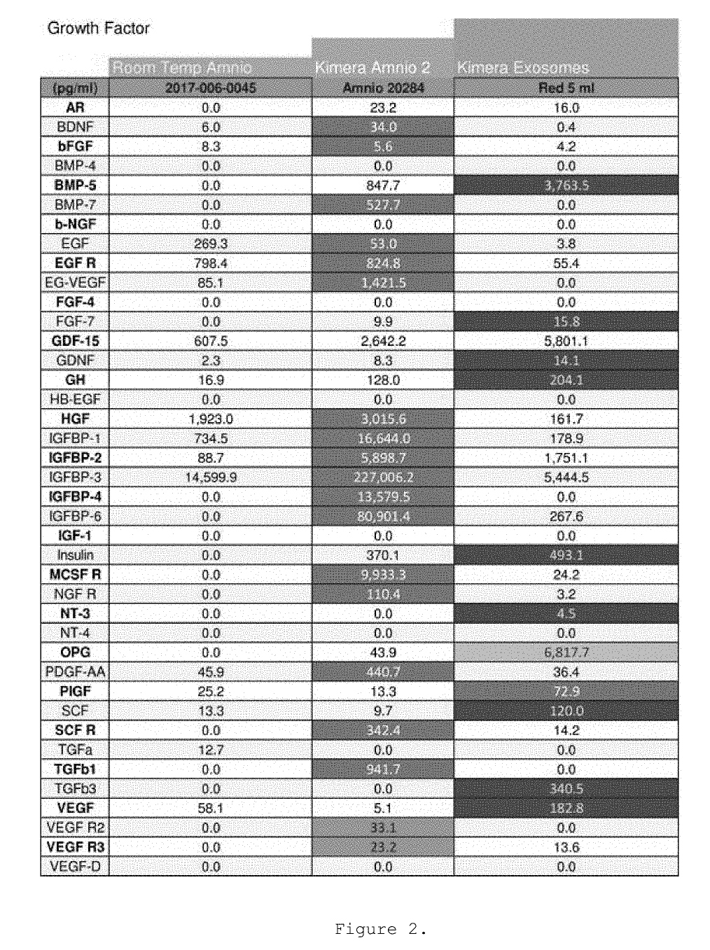

In one embodiment of the present disclosure, cell secretions in the form of microvesicles carry growth factors and a protein milieu that is distinct from cell to cell. The present invention uses cell secretory microvesicles derived from placental cotyledon derived mesenchymal stem cells that secrete a growth factor component outlined in FIG. 2, in concert with a phase 2 (Day 14) growth factor component from chondrocyte derived exosomes, to treat osteochondral defects and osteoarthritis.

To further the anti-inflammatory action of the first step of treatment, placental derived microvesicles are combined with cell free amniotic fluid, which provides a complimentary but dissimilar growth factor (FIG. 2, Amnio2) profile which can alternately be used to enhance the preliminary anti-inflammatory action in the synovial joint environment. Amniotic fluid has been used for centuries for its anti-microbial and skin replacement capabilities, and its addition to surgical protocols is warranted in almost every case.

It is known to those versed in the arts that mesenchymal stem cells (MSC) can persist for 7 days in cell culture within an initial fluid formulated to provide nutrients and growth factors. After this period the cells will exhaust the available resources or otherwise contaminate the system with cellular waste. In order to provide sufficient time for cell growth to occur and inflammation to be arrested, implanted cells or resident cells that have been induced to grow should be given a period of two weeks to expand in situ. During these two weeks, the cells will require placental exosome derived growth factors as listed in FIG. 2, including VEGF and PDGF for neovascularization and TGFB3 for anti-inflammatory function. They will also benefit from TNFRI and II for anti-inflammatory function and HGF and PDGF from amniotic fluid to induce growth (FIG. 2). After 14 days of growth and anti-inflammatory preparation, the cells are then ready for a FGF1 through FGF9 heavy growth factor milieu as obtained from chondrocytes in culture.

In concert with the above treatment, the growth factors described in FIG. 2 should be targeted to the area of damage by using a fibrin gel as described in US patent application 20150182558. This method has been demonstrated to improve cartilage defects in pre-clinical literature (Zhu 2017 Nanoscale) and fibrin has been shown to induce MSC to a chondrocyte phenotype.

In a further embodiment, Solution A can be modified with a locked nucleic acid of sequence +C+T+T+C+A+A+C+T+G+G+C+A+G+C+T, to enhance its anti-inflammatory effect.

In one embodiment of the present disclosure, the autologous cellular product obtained on Day 0 of the treatment will be co-incubated with a stimulatory solution that will aid in engraftment and expansion of the newly transplanted cells in situ. In one embodiment of the invention, the day 0 stimulatory Solution A will be comprised of microvesicles obtained from the supernatant of cultured placental decidua derived mesenchymal stem cells mixed in a 1:1, 1:2, or 1:3 ratio with allogeneic amniotic fluid or amniotic fluid derived microvesicles that harbor a distinct growth and anti-inflammatory factor profile in order to enhance engraftment potential, as described in FIG. 2.

In one embodiment of the present disclosure, the autologous cellular product of Day 0 will comprise in situ resident cells already present in the joint which will be co-incubated with a stimulatory solution comprised of stem cell derived exosomes, amniotic fluid, or a mixture thereof that will aid in engraftment and expansion of the newly transplanted cells in situ.

In one embodiment of the present disclosure, the anti-inflammatory and engraftment stimulatory solution (Solution A) may be accompanied by a distinctive color coding such as (for example) red labeling and/or packaging. The red coloration should assist the practitioner to distinguish the anti-inflammatory solution (needed for the initial cell differentiation and growth stage of the transplanted cells, which prefer a non-inflamed environment) from the Solution B more focused upon the later cellular differentiation and growth needs.

In one embodiment of the present disclosure, Solution A (red packaging) will be administered on one, two or three of the following days −14, −13, −12, −11, −10, −9, −8, −7, −6, −5, −4, −3, −2, −1,0,1,2,3,4,5,6,7,8,9,10,11,12,13,14.

In one embodiment of the present disclosure, Solution A (red packaging) will be administered between 1-24 hours prior to deployment of MSC cells into the patient's joint.

In one embodiment of this disclosure, Solution A comprises membrane-enclosed vesicles from one or more (i.e., a mixture) of cells selected from mesenchymal stem cells, amnion-derived multipotent progenitor cells, chorion derived mesenchymal stem cells, induced pluripotent stem cells, keratinocytes, fibroblasts, embryonic stem cells, ectodermal stromal cells, endodermal stromal cells, neural stem cells, proximal tubular epithelial cells, kidney mesenchymal stem cells, lung mesenchymal stem cells, lung epithelial cells, chondrocytes, tenocytes, hepatocyte progenitor cells, olfactory ensheathing cells, dental pulp stem cells and immortalized mesenchymal stem cells, or combinations thereof.

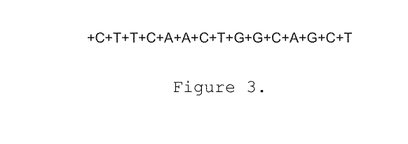

In one embodiment of the current disclosure, Solution A contains a locked nucleic acid with a sequence (SEQUENCE ID NO: 1) described in FIG. 3.

In one embodiment of this disclosure, Solution A comprises membrane-enclosed vesicles from one or more (i.e., a mixture) of cells and amniotic fluid.

In one embodiment of this disclosure, Solution A includes human allogeneic amniotic fluid.

In a certain embodiment, the membrane-enclosed vesicles are from mesenchymal stem cells. In another embodiment, the membrane-enclosed vesicles are from human cells.

In one embodiment of the present disclosure, Solution A may comprise a pharmaceutical composition of one or more growth factors, nucleic acids or chemicals or substances selected from the group consisting of GDF-1, FGF-1, IGF-1, IGF-1R, TGF-b, TGF-b2, TGFb3, EGF, miR-133b, bFGF, TIMP1, TIMP2, TIMP3, TIMP4, Wnt4 (protein or mRNA), angiopoietin, PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, fibronectin, Hsp90, gp96, myosin, keratin, annexin I, aldehyde dehydrogenase, ATP synthase, insulin like growth factor binding protein 1, GM-CSF, IGF like family member, miR-7, miR-13, miR-22, miR-26a, miR-27, miR-29, miR-29a, miR-30a, miR-100, miR-103, miR-106, miR-107, miR-122, miR-133, miR-140, miR-142-3p, miR-155, miR-210, miR-411, miR-483-5p, antago-miR-483-5p, miR-502-5p, miR-FOXP3, GDNF, hydrocortisone, Bacitracin, Neomycin Sulfate, Polymyxin B Sulfate, Pramoxine HCL, silver sulfadiazine, calendula, SEQUENCE ID NO: 1, citric acid, sodium chloride,GSK-3787, TLR3, TLR4, quercetin, indomethacin, insulin, dexamethasone, IBMX, rosiglitazone, ascorbate-2-phosphate, selenious acid, transferrin and sodium pyruvate, and mixtures thereof. The common functionality shared by the members of this group are that each is known to have immunomodulatory or anti-bacterial effects.

In one embodiment of the present disclosure, a distinct solution from Solution A, termed Solution B, will be administered on Day 14 or 15 or 16 or 17 or 18 or 19 or 20 or 21 or later. In another embodiment of the present disclosure, Solution B is accompanied by a distinctly different coloration from that of Solution A, such as (for example) a green syringe, labeling or packaging.

In one embodiment of the present disclosure, Solution B will comprise a pharmaceutical composition intended to cause terminal chondrocyte differentiation of cells affected by Solution A. Solution B may comprise (include) one or more growth factors, nucleic acids or chemicals or substances selected from the group consisting of GDF-1, FGF-1, FGF-2, FGF-3, FGF-4, FGF-5, FGF-6, FGF-7, FGF-8, FGF-9, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-13, miR-22, miR-26a, miR-27, miR-29, miR-29a, miR-30a, miR-100, miR-103, miR-106, miR-107, miR-122, miR-133, miR-140, miR-142-3p, miR-155, miR-210, miR-411, miR-483-5p, miR-502-5p, miR-FOXP3, GDNF, Hydrocortisone, Bacitracin, Neomycin Sulfate, Polymyxin B Sulfate, Pramoxine HCL, silver sulfadiazine, calendula, SEQUENCE ID NO: 1, citric acid, sodium chloride, GSK-3787, TLR3, TLR4, quercetin, indomethacin, insulin, dexamethasone, IBMX, rosiglitazone, ascorbate-2-phosphate, selenious acid, transferrin, and sodium pyruvate.

In one embodiment of this disclosure, Solution B comprises membrane-enclosed vesicles from one or more (a mixture) of cells selected from a chondrocyte, mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, neural stem cell, lung epithelial cell, chondrocyte, hepatocyte progenitor cell, olfactory ensheathing cell, dental pulp stem cell, or immortalized mesenchymal stem cell. In a certain embodiment, the membrane-enclosed vesicle is from a mesenchymal stem cell. In another embodiment, the membrane-enclosed vesicle is from a human cell.

In one embodiment of the present disclosure the source cell has been modified to overexpress the protein C-myc.

In one embodiment of the present disclosure, the method of treating and/or reducing inflammation comprises administering membrane-enclosed vesicle, wherein the membrane-enclosed vesicle is an endosome, an exosome and/or a microvesicle. In another embodiment, the membrane of the membrane-enclosed vesicle is from the plasma membrane. In a further embodiment, the plasma membrane-enclosed vesicle is substantially free of major histocompatibility complex (MCH).

In one embodiment of the present disclosure, the method of treating and/or reducing inflammation comprises administering membrane-enclosed vesicle, wherein the membrane-enclosed vesicle is about 10 nm to about 200 nm in diameter. In another embodiment, the membrane-enclosed vesicle is about 30 nm to about 100 nm in diameter.

In one embodiment of the present disclosure, the method of treating and/or reducing inflammation comprises administering a pharmaceutical composition comprising membrane-enclosed vesicles and one or more pharmaceutical acceptable carriers.

In one embodiment of the present disclosure, the method of treating and/or reducing inflammation comprises administering an effective amount of pharmaceutical composition comprising membrane-enclosed vesicles and additional therapeutic agent(s). In another embodiment, a pharmaceutical composition comprising membrane-enclosed vesicles comprises one or more growth factors, nucleic acids or chemicals selected from the group consisting of GDF-1, FGF-1, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-13, miR-22, miR-26a, miR-27, miR-29, miR-29a, miR-30a, miR-100, miR-103, miR-106, miR-107, miR-122, miR-133, miR-140, miR-142-3p, miR-155, miR-210, miR-411, miR-483-5p, miR-502-5p, miR-FOXP3, GDNF, Hydrocortisone, Bacitracin, Neomycin Sulfate, Polymyxin B Sulfate, Pramoxine HCL, silver sulfadiazine, calendula, SEQUENCE ID NO: 1, citric acid, sodium chloride,GSK-3787, TLR3, TLR4, quercetin, indomethacin, insulin, dexamethasone, IBMX, rosiglitazone, ascorbate-2-phosphate, selenious acid, transferrin, and sodium pyruvate.

In one embodiment of the present disclosure, the method of treating osteoarthritis (OA) comprises administering membrane-enclosed vesicles in one or more dosage forms selected from the group consisting of a solid dosage form, a cream, an aqueous mixture, a lyophilized aqueous mixture, and combinations thereof. In another embodiment, the membrane-enclosed vesicles are administered orally, intravenously or by inhalation, or combinations thereof. In a further embodiment, the membrane-enclosed vesicles are administered with the use of a nebulizer.

In one embodiment of the present disclosure, the method of treating and/or reducing inflammation in OA comprises administering an effective amount of a pharmaceutical composition comprising membrane-enclosed vesicles and one or more pharmaceutical acceptable carriers.

### Method of Amniotic Fluid

Amniotic Fluid (AF) for OA treatment is obtained from healthy c-section donors. AF is filtered via vacuum or gravity through a greater than 200 micron filter, prior to filtration through a 100 micron filter, prior to filtration through a 5 micron filter, and ultimately filtration through a 0.22 micron filter. AF is immediately frozen at −80 degrees celsius.

### Membrane-Enclosed Vesicles

Membrane-enclosed vesicles are extracellular or intracellular organelles which are enclosed by a lipid bilayer membrane, containing molecular constituents of the cell in which it originated from. For example, membrane-enclosed vesicles include exosomes, endosomes, microvesicles, liposomes, lysosomes, and the like. Some membrane-enclosed vesicles are extracellular, e.g., exosomes, and some membrane-enclosed vesicles are intracellular, e.g., endosomes. Extracellular membrane-enclosed vesicles carry and transfer molecules and other cellular content from one cell to another by a process commonly known as membrane vesicle trafficking. This process is believed to influence many biological and cellular processes, including the immune system.

### Exosomes

Exosomes are formed when secreted by the cells in which it originated from and contains, for example, cell-specific proteins, lipids, and genetic materials. Exosomes are found in many biological fluids, including blood, urine, and cell culture medium. It is understood that exosomes play an important role in intercellular signaling and communication, coagulation, as well as waste management (Raposo, G. et al. J. Cell Biol. 2013, 200, 373-383).

Exosomes are small in size with a range of diameters between about 2 nm and about 200 nm. Exosomes may have a range of size of diameters, such as between 2 nm to 20 nm, 2 nm to 50 nm, 2 nm to 100 nm, 2 nm to 150 nm or 2 nm to 200 nm. Exosomes may have a range of size of diameters, such as between 10 nm to 20 nm, 10 nm to 50 nm, 10 nm to 100 nm, 10 nm to 150 nm or 10 nm to 200 nm. Exosome may have a range of size of diameters between 20 nm to 50 nm, 20 nm to 100 nm, 20 nm to 150 nm or 20 nm to 200 nm. Exosomes may have a range of size of diameters, such as between 30 nm to 50 nm, 30 nm to 100 nm, 30 nm to 150 nm or 30 nm to 200 nm. Exosomes may have a range of size of diameters, such as between 50 nm to 100 nm, 50 nm to 150 nm or 50 nm to 200 nm. Exosomes may have a range of size of diameters, such as between 100 nm to 150 nm or 100 nm to 200 nm. An Exosome may have a size of a diameter between 150 nm to 200 nm.

The size of an exosome may be determined by various means known in the art. For example, the size of the exosome may be determined by size fractionation and filtration through a membrane with the relevant size cut-off and determined by tracking segregation of component proteins with sodium dodecyl sulfate polyacrylamide gel electrophoresis (SDS-PAGE) or by a biological assay. Alternatively, the size may also be determined by electron microscopy.

### Preparation and Isolation of Exosomes

Exosomes may be prepared and/or isolated in a variety of ways. In one embodiment, a method involves isolating exosomes from mesenchymal stem cells (MSC). MSC may be prepared by an in vitro proliferation of cell culture, for example, by dispersing an embryonic stem cell colony. Other cells in which exosomes can be isolated include, but are not limited to, amnion-derived multipotent progenitor cells, chorion derived mesenchymal stem cells, induced pluripotent stem cells, keratinocytes, fibroblasts, embryonic stem cells, ectodermal stromal cells, endodermal stromal cells, olfactory ensheathing cells, dental pulp stem cells, and immortalized mesenchymal stem cells.

Isolation of the exosomes from MSC may be done in a mesenchymal stem cell conditioned medium (MSC-CM). The MSC-CM may be obtained by culturing MSC, descendants thereof or a cell line derived therefrom in a cell culture medium and isolating the cell culture medium. The MSC-CM may be filtered and/or concentrated during, prior to and/or subsequent to separation. The MSC-CM may be filtered through a 0.22 micron membrane which has a particular porous size or a particular molecular weight cut-off of greater than 10 kilodalton. It may be subject to tangential force filtration or ultrafiltration.

Exosomes may also be synthesized or manufactured artificially, i.e., not isolated from a human or non-human cell. Instead of being isolated, exosomes could be synthesized by various lipid formation technologies.

Exosomes isolated from a human or non-human cell, or synthesized, can also be modified as needed for a particular treatment and/or use. For example, biomolecules such as messenger RNA, micro RNA, proteins or growth factors may be inserted (or removed) where desired. In one embodiment, one or more biomolecules may be exogenous, i.e., are not normally found in exosomes secreted by said cell type.

In one embodiment, 1,25-dihydroxycholecalciferol, BMP-1, Cadherin 11, KDR, collagen type I, collagen type II, collagen type III, collagen type IV, collagen type V, collagen type VI GDF-1, EGF, FGF-1, FGF-6, osteonectin, enolase 2, enolase 1, SDF-1, CSF-1, CSF-2, CSF-3, LIF-1, b-glycerophosphate, fibrillin 1, fibrillin-2, HSP-70, TGF-b, TGF-b2, TGFb3, EGF, ILF, miR-133b, bFGF, TIMP1, TIMP2, TIMP3, TIMP4, Wnt4 (protein or mRNA), PDGF-AA, PDGF-BB, G-CSF, VEGF, PGK, Kit-ligand, MCP-1, IL-6, IL-7, IL-8, IL-10, IDO IL-16, BMP1, MCP-1, RANTES, BDNF, HGF, KGF, procollagen, IFNg, MMP-1-9, E-cadherin, fibronectin, Hsp90, gp96, myosin, keratin, annexin I, aldehyde dehydrogenase, ATP synthase, insulin like growth factor binding protein 1, RANKL, GM-CSF, IGF like family member, miR-7, miR-100, miR-103, miR-106, miR-107, FOXP3, magnesium, zinc, boron, iron, fluoride, copper, vitamins A, K, E, D and C and/or GDNF messenger RNA of (Oct3/4, Sox2, Klf4, c-Myc) or miRNA targeting the expression of (Oct3/4, Sox2, Klf4, may be included and thus encapsulated by the exosomes.

Different physical or biological properties of the exosome may be used to separate the exosome from other components of MSC or MSC-CM, for example, based on molecular weight, size, shape, composition or biological activity. For example, high performance liquid chromatography (HPLC) with various columns may be used for separation of the exosomes. The columns may be size exclusion columns or binding columns. The monitoring of the exosomes during preparation and/or separation processes in MSC-CM may be carried out using, for example, light scattering, refractive index, fluorescently labeled antibodies, dynamic light scattering or UV-visible detectors. Similarly, other types of membrane-enclosed vesicles or compositions comprising said vesicles may be prepared and/or isolated by methods described herein or by methods commonly known in the art.

### Membrane-Enclosed Vesicle Compositions for Therapeutic Use

In one embodiment, the membrane-enclosed vesicle composition may be useful for the treatment of diseases or conditions associated with inflammation. In some embodiments, the membrane-enclosed vesicle composition may be useful for reducing the incidence of inflammation, modulating inflammation, or preventing inflammation. Non-limiting examples of inflammatory conditions include osteoarthritis (OA).

In one embodiment, the membrane-enclosed vesicle composition may be useful for the treatment of osteoporosis. Osteoporosis is a degenerative disease of the bones that strikes older patients, diabetic patients and post-menopausal patients and is caused by an imbalance between bone resorption and bone formation. Mesenchymal stem cells are capable of differentiation into bone, cartilage or adipose tissue. Microvesicles secreted from MSC have been shown to contain BMP-1 RANKL, and Cadherin 11 which have been shown to stimulate bone formation. Patients with inflammatory diseases prescribed glucocorticoids often experience decreases in bone mineral density over the course of treatment. In one embodiment, the membrane-enclosed vesicle composition is useful for the treatment of inflammation as a result of osteoporosis.

In some embodiments, the composition comprising membrane-enclosed vesicle may be an allogenic composition. That is, the membrane-enclosed vesicle to be administered to a subject is obtained from a different subject, but in the same group of species. For example, in a human subject, the membrane-enclosed vesicle is obtained or cultured from a different individual than those receiving the membrane-enclosed vesicle for therapeutic use.

The membrane-enclosed vesicle composition may be obtained from a variety of cell types. Particularly, one or more cells selected from of the group consisting of mesenchymal stem cells, chondrocytes, amnion-derived multipotent progenitor cells, chorion derived mesenchymal stem cells, induced pluripotent stem cells, keratinocytes, fibroblasts, embryonic stem cells, ectodermal stromal cells, endodermal stromal cells, olfactory ensheathing cells, dental pulp stem cells, and immortalized mesenchymal stem cells may be useful in harvesting, obtaining, and/or culturing membrane-enclosed vesicle compositions. In one embodiment, the membrane-enclosed vesicle composition is obtained from mesenchymal stem cells.

In one embodiment, the method of culturing the cells for the production of membrane-enclosed vesicles may further involve inducing oxidative stress. The oxidative stress may be induced by an externally added cytokine or by an oxidant such as hydrogen peroxide.

The membrane-enclosed vesicle compositions obtained by any of the processes described herein may be purified and isolated to obtain a composition that is concentrated in particular type(s) of membrane-enclosed vesicle(s). Alternatively, the membrane-enclosed vesicle composition obtained by any of the processes described herein, may be used without separating the different types of membrane-enclosed vesicles contained within.

In one embodiment, the membrane-enclosed vesicles are selected from one or more of endosome, exosome, and microvesicle. In another embodiment, the membrane-enclosed vesicles comprise plasma membrane as the enclosure membrane. In one embodiment, the membrane-enclosed vesicles are derived from the plasma membrane. In a certain embodiment, the plasma membrane-enclosed vesicles are substantially free of major histocompatibility complex (MHC).

In one embodiment, the membrane-enclosed vesicles useful for therapeutic purposes have a diameter range of about 10 nm to about 200 nm and are supplied at a concentration of 1 mg/ml or greater. In another embodiment, the diameter range of the membrane-enclosed vesicle is about 30 nm to about 100 nm.

In some embodiments, the cells for producing membrane-enclosed vesicles may be obtained from a human subject. In one embodiment, the cells may be obtained from a human subject who is between 1 to 12 months old. In other embodiments, the cells may be obtained from a human subject who is about 1 to 30, 35, 40, 45, or 50 years old. In another embodiment, the cells may be obtained from a human subject who is greater than 50 years old.

### Pharmaceutical Composition Comprising Membrane-Enclosed Vesicle

The membrane-enclosed vesicle composition obtained by various methods disclosed herein, in one embodiment, may be formulated with one or more pharmaceutical acceptable carrier, excipient, adjuvant, diluent and/or binder. Suitable pharmaceutically acceptable carriers, excipients and diluents may include one or more of any and all conventional solvents, dispersion media, fillers, solid carriers, aqueous solutions, coatings, vehicles suitable for topical administration, other antimicrobial agents, isotonic and absorption enhancing or delaying agents, activity enhancing or delaying agents for pharmaceutically active substances, and are well known in the art. Common pharmaceutically acceptable additives are disclosed, by way of example, in Remington: the Science & Practice of Pharmacy by Alfonso Gennaro, 20^{th} ed., Lippencott Williams & Wilkins, (2000). Except insofar as any conventional carrier, excipient or diluent incompatible with the membrane-enclosed vesicle composition, use thereof in the present invention is contemplated.

In one embodiment, the carrier is a fibrin gel derived from the patient's own blood to facilitate extended release of the administered microvesicles.

In one embodiment, the membrane-enclosed vesicle composition may have one or more externally added additional, compatible, pharmaceutically-active materials. In a certain embodiment, the membrane-enclosed vesicle composition comprises externally added one or more growth factors, nucleic acids, or protein molecules. In some embodiments, the membrane-enclosed vesicle composition comprises one or more growth factors selected from the group consisting of 1,25-dihydroxycholecalciferol, GDF-1, FGF-1, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, PGK, MCP-1, IL-6, IL-7, IL-8, IL-10, IDO IL-16, BMP1, BDNF, HGF, KGF, IFNg, MMP-1-9, E-cadherin, Fibronectin, Hsp90, gp96, Myosin, Keratin, Annexin I, aldehyde dehydrogenase, ATP synthase, insulin like growth factor binding protein 1, GM-CSF, IGF like family member, miR-7, miR-100, miR-103, miR-106, miR-107, FOXP3, RANKL, and GDNF.

### Administration of Membrane-Enclosed Vesicle Composition

One embodiment of the present invention included methods for treating various conditions by administering a therapeutically effective amount of a membrane-enclosed vesicle from a cell.

In a specific embodiment, the method may include treating osteoarthritis (OA). In a specific embodiment, the methods for treating OA may be by administering to a patient in need thereof a therapeutically effective amount of a membrane-enclosed vesicle from a cell. In a specific embodiment, the cell is selected from one or more of the group consisting of a mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, olfactory ensheathing cell, dental pulp stem cell, and immortalized mesenchymal stem cell. In another embodiment, the cell is a mesenchymal stem cell. In another embodiment, the cell is a human cell. In another embodiment, the cell is a chondrocyte.

In another embodiment of the methods of the present invention, the plasma membrane is substantially free of major histocompatibility complex (MHC).

In one embodiment of the invention, the microvesicle preparation is infused into the intraarticular space prior to infusion of a fibrin gel matrix or cells.

In another embodiment of the methods of the present invention, the membrane-enclosed vesicle is an endosome, an exosome and/or a microvesicle. In a specific embodiment, the membrane of the enclosed vesicle is from the plasma membrane. In another embodiment, the membrane-enclosed vesicle is about 10 nanometers to about 200 nanometers in diameter. In another embodiment, the membrane-enclosed vesicle is about 30 nanometers to about 100 nanometers in diameter.

In another embodiment, the membrane-enclosed vesicle is administered to a patient in one or more dosage forms selected from the group consisting of a solid dosage form, a cream, an aqueous mixture and a lyophilized aqueous mixture. In another embodiment, the membrane-enclosed vesicle is administered orally, intravenously or by inhalation. In another embodiment, the membrane membrane-enclosed vesicle is administered in a pharmaceutical composition comprising one or more pharmaceutical acceptable carriers.

In another embodiment, the membrane-enclosed vesicle comprises one or more growth factors (outlined in FIG. 2) selected from the group consisting of GDF-1, FGF-1,FGF-9, FGF-6, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-100, miR-103, miR-106, miR-107, FOXP3, and GDNF. The common functionality shared by the members of this group is known to have immunomodulatory or anti-bacterial effects. In a specific embodiment at least one of the one or more growth factors are exogenous to the cell and/or the membrane-enclosed vesicle.

In another embodiment, the membrane enclosed vesicle is administered bound in a fibrin clot.

In another embodiment, the membrane enclosed vesicle includes an oligonucleotide with a nucleic acid sequence described in SEQUENCE ID NO: 1.

In another embodiment, the membrane-enclosed vesicle comprises one or more microRNA (miRNA) selected from the group consisting of miR-4^{a},miR-8,miR 9,miR 16,miR-17-3p,miR-19a-3p, miR-19b3p miR-21,miR 21,miR 21-5p,miR 26^{a},miR 24,miR 27b,miR 27b,miR 28-5p,miR-29^{a},miR29b,miR29c,miR 30^{a},miR-30a-3p,miR 30e,miR 31,miR 34^{a},miR 34a-5p,miR-92^{a},miR-93,miR 101,miR 106^{a},miR 106b,miR 122,miR 124,miR-124^{a},miR 124-3p,miR125,miR 125b,miR126-IKB-a,miR-127miR 129,miR 130^{a},miR 130b,miR132,miR-135p,miR 137,miR 141,miR 141^{a},miR 143,miR-144,miR 145,miR-146^{a},miR 147,miR-148,miR-148b,miR 151,miR 155,miR 181,miR-181b,miR 182,miR 184,miR 185,miR-191,miR 192,miR 194,miR -196^{a},miR-197.miR 200,miR 200^{a},miR 200b,miR-200c,miR 203,miR 204,miR 205,miR-207,miR-214,miR 221,miR-222,miR-222-3p,miR-223,miR-223-3p,miR 301^{a},miR-320,miR 323-3p,miR-324-5p-CUEDC2,miR-328,miR-339-5p,miR-355,miR 379-5p,miR 382,miR-409-3-p,miR-422a,miR 424,miR-429,miR 431,miR 449,miR-449^{a},miR 466g, miR-483-5-p, antago-miR-483-5-p miR 483-3p,miRNA 487b,miR 488,miR 489,miR 494,miR-495, miR 497,miR 509-3p,miR 610,miR 663,miR 671,miR 887 ,miR-1180,miR -1224-5p,miR 1290, miR 1246, miR-1271, miR-1826,miR-3473a,miR3619, miR-5128, and miR-6500-3p. In one embodiment, the pharmaceutical dosage form comprising the membrane-enclosed vesicle is formulated as a sustained-release system. A non-limiting example a sustained-release formulation is a semi-permeable matrix of solid hydrophobic polymers. In certain embodiments, sustained-release systems may, depending on their chemical nature, release pharmaceutical agents over a period of hours, days, weeks or months.

The membrane-enclosed vesicle composition may have a concentration of membrane-enclosed vesicle that are about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 ng/ml, μg/ml, mg/ml, or g/ml, or any range derivable therein.

The membrane-enclosed vesicle composition may be administered to or self-administered by the subject 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein.

All examples and conditional language recited herein are intended for educational purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents hereof. Additionally, it is intended that such equivalents include both currently known equivalents as well as equivalents developed in the future, i.e., any elements developed that perform the same function, regardless of structure.

We claim:

1. A method of treating a subject's osteochondral defects comprising the steps of:
(a) administering an effective amount of anti-inflammatory cell growth pharmaceutical composition into a joint cavity; and
(b) administering into said joint cavity an effective amount of pharmaceutical composition for cell differentiation and growth.

2. The method of claim 1, said step (a) of administering an effective amount of anti-inflammatory pharmaceutical composition into a joint cavity comprising transplanting a solution of said cells into a joint space.

3. The method of claim 2, wherein said anti-inflammatory pharmaceutical composition comprises cells having the locking nucleic acid sequence of +C+T+T+C+A+A+C+T+G+G+C+A+G+C+T.

4. The method of claim 1, said anti-inflammatory pharmaceutical composition comprising membrane-enclosed vesicles derived from cells of the group consisting of mesenchymal stem cells, amnion-derived multipotent progenitor cells, chorion derived mesenchymal stem cells, induced pluripotent stem cells, keratinocytes, fibroblasts, embryonic stem cells, ectodermal stromal cells, endodermal stromal cells, neural stem cells, proximal tubular epithelial cells, kidney mesenchymal stem cells, lung mesenchymal stem cells, lung epithelial cells, chondrocytes, tenocytes, hepatocyte progenitor cells, olfactory ensheathing cells, dental pulp stem cells and immortalized mesenchymal stem cells, or combinations thereof.

5. The method of claim 4, said anti-inflammatory pharmaceutical composition comprising membrane-enclosed vesicles comprising secretory membrane-enclosed vesicles derived from placental-derived mesenchymal stem cells.

6. The method of claim 5, said anti-inflammatory pharmaceutical composition further comprising amniotic fluid.

7. The method of claim 6, said membrane-enclosed vesicles comprised of microvesicles obtained from the supernatant of cultured placental decidua derived mesenchymal stem cells mixed with amniotic fluid in a ratio of between about 1:1 to 1:3.

8. The method of claim 6, said anti-inflammatory pharmaceutical composition further comprising placental exosome-derived growth factors.

9. The method of claim 1, wherein step (a) further comprises the preparatory step of encapsulating said cells in fibrin gel.

10. The method of claim 1, said step (a) administration occurring during the first 13 days after commencement of treatment.

11. The method of claim 1, wherein said anti-inflammatory cell growth pharmaceutical composition comprises a slow release pharmaceutical composition that comprises a unit dose of one or more growth factors and/or nucleic acids on at least three of the following days: −14, −13, −12, −11, −10, −9, −8, −7, −6, −5, −4, −3, −2, −1,0,1,2,3,4,5,6,7,8,9,10,11,12,13 and 14.

12. The method of claim 1, wherein said anti-inflammatory cell growth pharmaceutical composition comprises a therapeutically effective amount of cells comprising between about 30,000 and about 500,000 cells including CD90+CD105+CD73+MSC; and
wherein said step (b) of administering an effective amount of pharmaceutical composition for cell differentiation and growth comprising injecting growth factor milieu into the joint cavity; and
wherein said composition comprising growth factors obtained from cultured chondrocytes.

13. The method of claim 1, wherein said pharmaceutical composition for cell differentiation and growth comprises a slow release composition that comprises a unit dose selected from the group consisting of GDF-1, FGF-1, TGF-b, TGF-b2, TGFb3, EGF, miR-133b, bFGF 1-9, TIMP1, TIMP2, TIMP3, TIMP4, Wnt4 (protein or miRNA), PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-13, miR-22, miR-26a, miR-27, miR-29, miR-29a, miR-30a, miR-100, miR-103, miR-106, miR-107, miR-122, miR-133, miR-140, miR-142-3p, miR-155, miR-210, miR-411, miR-483-5p, miR-502-5p, miR-FOXP3, GDNF, hydrocortisone, Bacitracin, Neomycin Sulfate, Polymyxin B Sulfate, Pramoxine HCL, silver sulfadiazine, calendula, SEQ ID NO: 1, citric acid, sodium chloride,GSK-3787, TLR3, TLR4, quercetin, indomethacin, insulin, dexamethasone, IBMX, rosiglitazone, ascorbate-2-phosphate, selenious acid, transferrin and sodium pyruvate, or combinations thereof.

14. The method of claim 1, said step (b) occurring subsequent to day 13 after commencement of treatment; and
wherein step (b) further comprises the preparatory step of encapsulating said cells in fibrin gel; and
wherein said pharmaceutical composition for cell differentiation and growth comprises a slow release pharmaceutical composition that comprises a unit dose of 5 to 10,000 mg of anti-inflammatory membrane enclosed vesicles; and
wherein the pharmaceutical composition is of immediate release; and
wherein either step of administration includes hyaluronic acid.

15. A method of claim 1 further comprising the step of administering an effective amount of a pharmaceutical composition altered to impart a cellular differentiation pathway to affected cells on day 13, 14, 15 or later of treatment; and
wherein step (b) is altered and administered on day 13, 14, 15 or later of the treatment to include growth factors selected from the group consisting of FGF-1,2,3,4,5,6,7,8 and 9, or combinations thereof and
wherein step (b) is altered on day 13, 14, 15 or later of the treatment to include a cellular differentiation medium selected from the group consisting of Transferrin-Selenium, dexamethosone, ascorbate-2-phosphate, transforming growth factor-beta 1, and sodium pyruvate, or combinations thereof.

16. The method of claim 1 wherein step (b) is altered to include a cellular differentiation medium including membrane enclosed vesicles derived from chondrocytes of fetal or neo-natal origin; and
wherein step (b) includes a unit dose of 5 to 3000 mg of membrane enclosed vesicles derived from a cell or chondrocyte of fetal or neonatal origin.

17. A method of producing a composition for treating osteochondral defects comprising the steps of:
(a) disassociating cells of placental cotyledon tissue;
(b) culturing said cells; and
(c) collecting exosomes from said culture.

18. The method of claim 17, said exosomes having a diameter in the range of between about 10 nm to about 200 nm and comprising lipid vesicle growth factors and wherein said exosomes having a diameter in the range of between about 30 nm to about 100 nm and comprising lipid vesicle growth factors.

19. A method of enhancing osteochondral tissue, comprising the steps of:
(a) administering into a joint cavity, in doses not more frequently than daily for up to 13 days, an effective amount of anti-inflammatory cell growth pharmaceutical composition comprising cell secretory microvesicles derived from placental cotyledon derived mesenchymal stem cells that secrete growth factors; and
(b) administering into said joint cavity on day 14 and as appropriate thereafter, an effective amount of pharmaceutical composition for cell differentiation and growth comprising chondrocyte derived exosomes.

20. The method of claim 19, wherein said step (b) administration of pharmaceutical composition for cell differentiation and growth is accompanied by a continuation of said step (a) administration of anti-inflammatory cell growth pharmaceutical composition.
